(19) United States
(12) Patent Application Publication
(10) Pub. No.: US 2017/0181663 A1
(43) Pub. Date: Jun. 29, 2017

(54) SYSTEM FOR MONITORING RESPIRATORY EFFORT

(71) Applicant: SPIRO MEDICAL AS, Bergen (NO)

(72) Inventors: Rolf Kahrs HANSEN, Bergen (NO); Magne TVINNEREIM, Bergen (NO); Regina CONRADT, Oxford (GB)

(73) Assignee: Spiro Medical AS, Bergen (NO)

(21) Appl. No.: 15/313,158

(22) PCT Filed: May 26, 2015
(86) PCT No.: PCT/EP2015/061541

§ 371 Date: Nov. 22, 2016

(30) Foreign Application Priority Data
May 26, 2014 (NO) 20140652
Aug. 22, 2014 (NO) 20141036

(51) Int. Cl. A61B 5/085; A61B 5/00; A61B 5/087; A61B 5/03; A61B 5/08

(52) U.S. Cl. CPC A61B 5/085; A61B 5/037; A61B 5/0816; A61B 5/0878; A61B 5/4818; A61B 5/0002; A61B 5/6823; A61B 5/6831; A61B 5/7282; A61B 5/7246; A61B 5/087

(57) ABSTRACT

System for monitoring respiratory effort in a patient, comprising at least one pressure sensor, the pressure sensor being positioned in a catheter and being adapted for positioning in a chosen position in the oesophagus, the pressure sensor being adapted to monitor the pressure difference between the oesophagus and external pressure at a chosen rate. The system comprises flow measuring means for measuring the respiratory flow and analyzing means for calculating the respiratory admittance or the respiratory impedance as well as detecting the respiratory related arousals.

The invention relates to a system for monitoring respiratory effort in a patient.

More specifically the invention relates to a system and method for recording and analysis of Sleep Related Breathing Disorders (SRDB). The system discriminates objectively between Central and Obstructive SRBDs. Obstructive SRBDs are found in Obstructive Sleep Apnea (OSA) and Upper Airway Resistance Syndrome (UARS) with Respiratory Effort Related Arousals (RERA). Central SRBDs are found in Central Sleep Apnea (CSA) and more commonly in Cheyne Stoke Respiration (CSR).

### BACKGROUND OF THE INVENTION

It is internationally recognized that “The measurement of oesophageal pressure with continuous overnight monitoring is the reference standard for measuring respiratory effort”, American Academy of Sleep Medicine (AASM) 2007 /1/ (see list of references below). Obstructive SRBD is a relatively newly discovered disease entity, characterized by snoring, daytime hypersomnolence and different degrees of impaired air passage (apneas and hypopneas) through a narrowed, relaxed upper airway during sleep /2/.

Investigations have shown that increased airway pressure during sleep along with snoring and arousals can result in health problems like those initiated by apneas and hypopneas /3/. The higher number of arousals and increased percentages of lighter sleep stages, the important brain and body rest functions are compromised together with other functions like the arterial oxygenation and heart rate /4/. The result may be multiple changes throughout the body, such as hypertension, heart arrhythmias, heart attack, hormone changes, brain hemorrhage as well as social disorders. There is also profound evidence of increased risk of involvement in traffic accidents. /5/. These problems are seen in addition to socially unacceptable snoring, which is in fact the most important reason for people to seek medical treatment.

Numerous approaches to detect arousals in sleep involve recording signals modulated by brainwave activity, muscle tone, or parameters of autonomic arousal response.

Today, arousal detection is either based on the American Academy of Sleep Medicine (AASM) criteria /9/ or on surrogate parameter. Traditionally an arousal is an event that occurs during sleep and usually is identified by changes in EEG signals during non-REM sleep and changes in EEG and EMG signals during REM sleep. Arousals usually do not turn into wakefulness and are not recognized by the individual. The “gold standard” in terms of accuracy for monitoring respiratory arousals is monitoring electrophysiological parameters (EEG, EMG, EOG) and respiratory parameters (usually by respiratory bands around the chest and abdomen and oro-nasal flow). This technique based on the AASM criteria is unsuitable for outpatient use. A full cardio-respiratory PSG has a number of disadvantages. It is mostly done in a specialized sleep laboratory within a hospital, it is cumbersome for the patient, time-consuming to set up and to analyze, requires a specialist technician, and inevitably increases the cost of the sleep study.

However, the arousals, as identified by changes in electrophysio logical signals, are a part of the activation of the autonomic nervous system (ANS). Not all transient activation of the ANS can be analysed by the AASM criteria. Our new system for diagnosing SRBD related Arousals will detect all Respiratory Effort related Arousal events. This includes both, standard AASM detected arousals as well as more subtle

ANS mediated arousals. This objective is obtained as specified in the independent claims.

The two most common sleep disorders, Obstructive Sleep Apnea (OSA) and movement disorder events, show frequent autonomic arousal events concurrent with the sleep disorder. In order to link the autonomic arousal to the specific disorder, like OSA, corresponding signals are recorded and combined.

The transient activation of the autonomic nervous system to detect respiratory arousals can be verified by various signals. Accelerometers may be used to detect patient's movement correlated to respiratory arousal /12/. An ECG or a pulse oximeter may be used to identify different cardiac variables related to transient activation. For example, heart rate increases upon arousal /15/, the Pulse wave amplitude (PAW) decreases upon arousal /18/, and heart rate variability is modified by autonomic tone changes associated with arousal /13/. Cardiac output increases during arousal and can be measured by noninvasive electrical impedance electrodes /14, 15, 16/. Blood pressure can be measured by a non-invasive pressure gauge /17/ and shows transient rises with arousal from sleep and each inspiration causes a transient blood pressure fall. The respiratory arousal detection can be based on attenuation of the Peripheral Arterial Tone (PAT) amplitude /11/. Pulse transit time (PTT) is a non-invasive technique which reflects changes in peripheral vascular resistance /10/ and is sensitive to detect respiratory arousals.

Arteriole size, which may be measured by photoplethysmography, decreases upon arousal due to sympathetic nervous system activation. Implantable devices can be coupled to microelectrodes measuring sympathetic nerve traffic during arousals /16/. To improve the quality of detection, combination of different parameters is a possibility. Hoffman /6/ teaches how to measure lung volume and flow simultaneously and combines these into one output parameter. Lung volume can be measured with respiration belts and flow can be measured using flow sensors, temperature variation or microphones. The output parameter can be a comparison of the two input signals, analysis of phase and magnitude differences in the same time domain. The author also teaches that the phase and magnitude differences can be used to determine the site of obstruction.

The present invention uses the variation of the relationship between pressure and flow. Other authors /22/ mention the relation between pressure and flow and use this as a means for detecting Inspiratory Flow Limitation (IFL) and differentiate between obstructive and central hypopneas. Our approach is to use the ratio between flow and pressure (the respiratory admittance) over time to detect arousals as explained in the following.

Visually, the changes of respiration during and after obstructive events are prominent features. While the changes in respiratory amplitude measured in respiratory flow have led to the definition of the events (a-pnoe meaning without breathing, and hypo-pnoe meaning reduced breathing), the more subtle changes in respiratory frequency were widely neglected in literature.

Bloch et al. /25/ looked at the respiratory pattern in snoring patients. A small part of their investigation involved the analysis of the pre- and post-arousal breathing pattern in habitual snorers. However, while they could find significant differences in some parameters of the respiratory pattern (e.g. tidal volume), they failed to show a difference in the respiratory frequency pre- and post-arousal. This could have several reasons, firstly the very small number of patients (8), secondly a small observation number of 40 and thirdly the observation method based on a defined number of breaths taken into account for the statistical analysis.

Ben-Israel et al. /26/ could show in 2012 that snoring can be used successfully to differentiate subjects according to AHI, using snoring variability and inter-event silence between two snoring events. To our knowledge, no published research has focused to include the snoring signal into the arousal detection.

The invention will be described in more detail below with reference to the accompanying drawings. Illustrating the invention by way of examples.

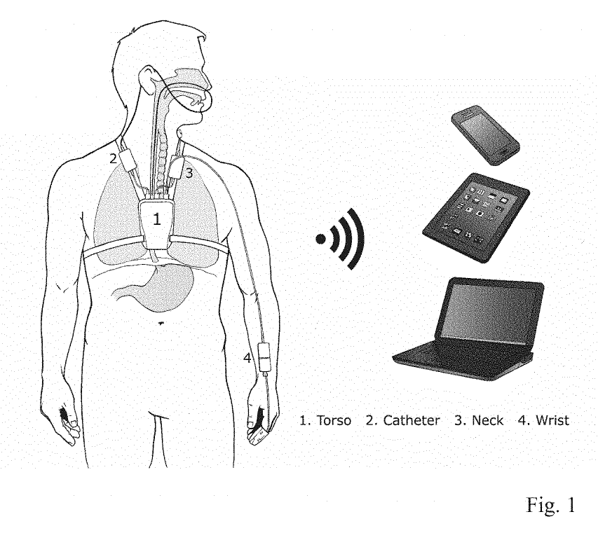

FIG. 1 illustrates the system according to a preferred embodiment.

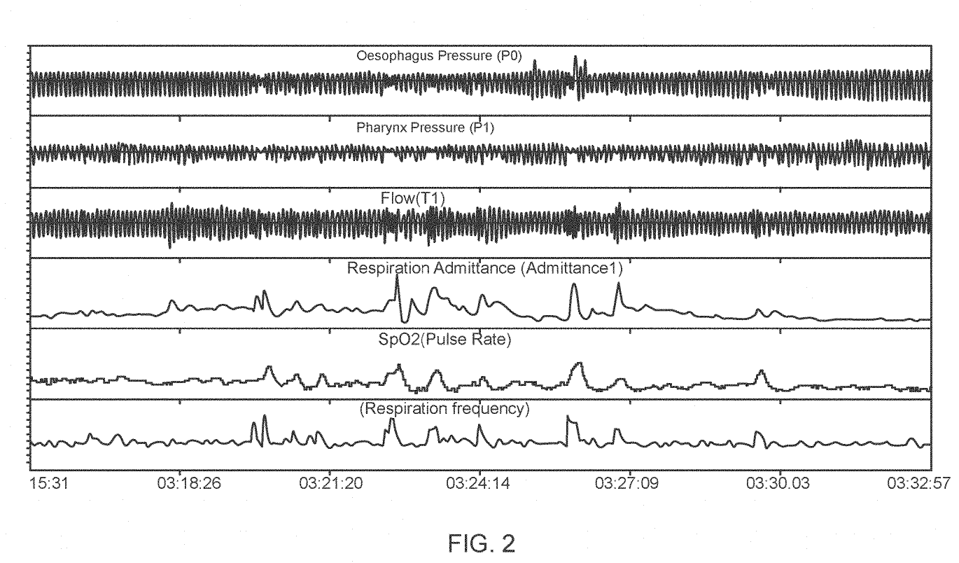

FIG. 2-8 illustrate simultaneous data indicating Oesophagus pressure, Pharynx pressure, flow, Respiratory admittance, SpO2 (Pulse rate) and respiration frequency.

The present invention may be described as
to provide methods of recording and analyzing respiratory pattern during sleep,
to provide methods of recording and analyzing Respiratory Effort Related Arousals (RERA),
to provide methods to verify the presence of a RERA taking into account additional parameters (e.g. pulse rate (PR), pulse transit time (PTT), respiratory rate or snoring).

The respiration is tightly regulated. The goal is to keep the Oxygen (pO2) and Carbon dioxide (pCO2) levels within tight ranges and to minimize the concurrent energy costs (muscle work). During obstructive breathing the upper airways become tightened or occluded, which leads to a reduced airflow to the lungs. A reduction in airflow leads to imbalances in the blood gases (increased pCO2, decreased pO2) and the body reacts with increasing respiratory effort. The body will trigger a sympathetic arousal reaction /19, 20/. The arousal reaction stimulates the pharyngeal muscle activity. The upper airway becomes more patent and can fulfill its respiratory goals. The airflow increases and can maintain the blood gases constant and the respiratory effort decreases, which reduces the energy costs of respiration /21/.

The present invention uses the esophageal pressure to represent lung pressure. This is considered as the force that drives the respiration. The resulting respiration is then the resulting flow. If the respiratory channel is open, without restrictions, air flows freely and only a minor pressure differential (lung pressure minus external pressure) is required to produce a significant flow. This means that the respiratory impedance is low- or that the respiratory admittance is high. If the respiratory channel is restricted, a higher pressure differential is required to produce the same flow. This means that the respiratory impedance is high- or that the respiratory admittance is low. In the following, we will follow the notations developed within electricity as these are well known and understood. Division of pressure with flow yields impedance while division of flow with pressure yields admittance:

$$\begin{matrix}Z = \frac{P}{F} & ( 1 ) \\ Y = \frac{F}{P} & ( 2 )\end{matrix}$$

Here, P is pressure in Pascal, F is flow in m3/s, Z is impedance and Y is admittance. Z and Y both have phase and amplitude and complex representations cover both phase and magnitude.

Z=R+jX (3)

y=G+jb (4)

Here, R is respiratory resistance and X is respiratory reactance, G is respiratory conductance and B is respiratory susceptance. j is the imaginary unit such that j²=−1.

Admittance and impedance are related as follows:

$$Y = {Z}^{- 1} = \frac{1}{R + jX} = ( \frac{1}{{R}^{2} + {X}^{2}} ) ( R - jX ) \qquad ( 5 )$$

Admittance phase and magnitude are obtained as follows:

$$\begin{matrix}∠ Y = arctan ( \frac{B}{G} ) = arctan ( - \frac{X}{R} ) & ( 6 ) \\ \vert Y \vert = \sqrt{{G}^{2} + {B}^{2}} = \frac{1}{\sqrt{{R}^{2} + {X}^{2}}} & ( 7 )\end{matrix}$$

Using admittance as the key parameter, a typical time series is illustrated in FIG. 1 showing an example of admittance and pulse rate during sleep with respiratory related arousals.

The sharp admittance peaks correspond to events where flow suddenly increases due to stimuli to the respiratory channel, forcing it open. This is a respiratory event triggered by the sympathetic nervous system and representing an arousal in the same way as PTT detection. As the figure shows, there is good correlation between pulse rate variation and respiration admittance. Pulse rate can thus be used to verify the arousal if such a sensor signal is available.

It is the change in admittance per time unit that signals a respiratory event, hence the scoring parameter is the time derivative of the admittance.

Typically, one should require a p % increase in admittance per respiratory cycle (typically 5 seconds) of the admittance in order to classify the event as an arousal. P would normally be 50%.

A RERA event is scored when the following occurs:
Increasing lung pressure for 10 seconds or more,
Admittance increase p % over one respiratory cycle.

Lung pressure can be measured as in one of the following ways:
a) Directly with a catheter with a pressure sensor in the oesophagus (as lung pressure can be represented by data obtained in the oesophagus /1/).
b) Indirectly from changes of the respiratory belts length (circumference) and calibrated against lung pressure for each individual,
c) Indirectly from respiratory belts stress and calibrated against lung pressure for each individual.

The preferred embodiment is a). It is direct, the sensor is well protected and can be calibrated accurately.

Flow can be measured as in one of the following ways:
a) Directly with a flow meter in a mask over the patient's nose and mouth.
b) Indirectly with an oral-nasal thermal sensor sensing the temperature difference between inhalation and exhalation.
c) Indirectly by sensing temperature in a catheter in the respiration channel, sensing the temperature difference between inhalation and exhalation.
d) Indirectly by measuring the acoustic flow noise with a sensitive contact microphone.
e) Indirectly by the use of a nasal cannula or an oral-nasal cannulas measuring pressure in the direction of the flow.

The preferred embodiment is c), especially in combination with oesophagus pressure measurement as they can use the same catheter. This method ensures that the sensor is protected and it cannot move if the catheter is properly secured. The use of dual sensors placed apart yields detection of nose flow as well as the combined mouth-nose flow. Note that although the relationship between temperature variation and flow is nonlinear and depends on the difference between body temperature and external temperature, it is still a good sensor when it is most important, when the flow is low or almost zero.

Abrupt changes in the flow are then easily and reliably detected.

Heart rate or pulse rate can be measured as in one of the following ways:
a) Directly with ECG electrodes on the chest,
b) Indirectly with oximeter based pulse rate sensor,
c) Indirectly with a contact microphone fitted on the chest or on a major blood vessel.

The preferred embodiment is b) as oxygen saturation is also normally measured and used in the diagnosis of the severity of OSAS, UARS or SRBD.

Other parameters that can be correlated with RERA and Respiratory Effort (RE):
Actimeter sensor on legs and /or arms,
Body position sensor,
Snoring measured with microphones in air or contact microphones.
Biopotential sensors (EMG, EEG, ECG)

A typical set of sensors is shown in FIG. 1. Here a data acquisition unit 1 at the torso is provided with a Bluetooth radio link to some nearby computer. A catheter sensor 2 is used for measurement of respiration flow and esophageal pressure, a wrist unit 4 is provided in order to measure pulse oximetry and actimetry. As illustrated the oximetry sensor may be positioned on the patient finger.

In addition, a microphone unit or similar 3 measures snoring sound. The snoring sound may be detected as the sound amplitude, possibly in a chosen frequency range, or to reduce the disturbance from other sounds in the environment by using more advanced recognition techniques.

The computer 5 may be of any available type, including smart phones or tablets, being capable of performing the analysis and presenting the results. Some analyses may also be performed in the torso unit 1 before communicating them to the computer. As mentioned the communication is preferably a wireless system, but cable connections or memory cards may also be used, depending on the required data rate and available system.

The torso unit may also store the data internally and data may be transferred to computer 5 via cable or wirelessly after the recording of data has been completed. In FIGS. 2-5 the measurements are illustrated, show from top to bottom:
The oesophagus pressure which represents the lung pressure (band pass filtered).
The temperature variation which represents the variation of temperature between inhalation and exhalation and thus represents flow.
The respiratory admittance as explained in the text.
The pulse rate measured with a finger SpO2 probe.
The respiration frequency derived from the oesophagus pressure.

FIGS. 2-5 show examples on how the admittance is a combination of oesophagus pressure and temperature variation due to flow. Arousals occur when there is an increasing pressure and low flow and then a sudden decrease in pressure combined with an increase of flow—causing an admittance peak. The arousal can be verified by an increase in pulse rate and an increase in respiratory rate.

FIG. 2 shows 18 min of a recording. The figure demonstrates that the Respiration admittance is calculated by a combination of oesophagus pressure and flow (temperature variation due to flow, T1). RERA (Respiratory Effort Related Arousal) events occur if a period of at least 10 seconds with increasing pressure and reduced flow is followed by a sudden decrease in pressure concurrent with an increase of flow. A sudden decrease in pressure concurrent with an increase of flow will be shown as a peak in respiration admittance. A RERA event is detected using the peaks in respiration admittance and is verified using pulse rate and respiration frequency

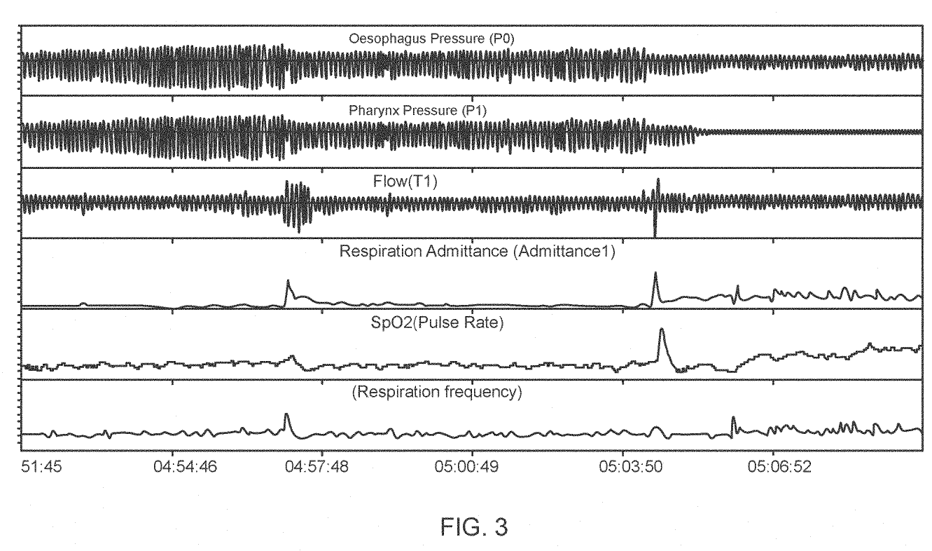

FIG. 3 shows two RERA events. The first RERA took place at around 04.57 the second at around 05.04. Prior to the RERAs both pressure signals, oesophagus as well as pharynx, showed increasing amplitudes. At 04.57 and at 05.04 the constant increase was suddenly interrupted by a distinct decrease in both pressure recordings concurrent with a rapid increase in flow (T1). Both times this was detected as a sharp increase in respiratory admittance. The peaks in respiration admittance coincided with an increase in pulse rate as well as a change in the respiratory pattern—here an increase in respiration frequency. A RERA event is detected using respiratory admittance and is verified using peaks in pulse rate and respiration frequency.

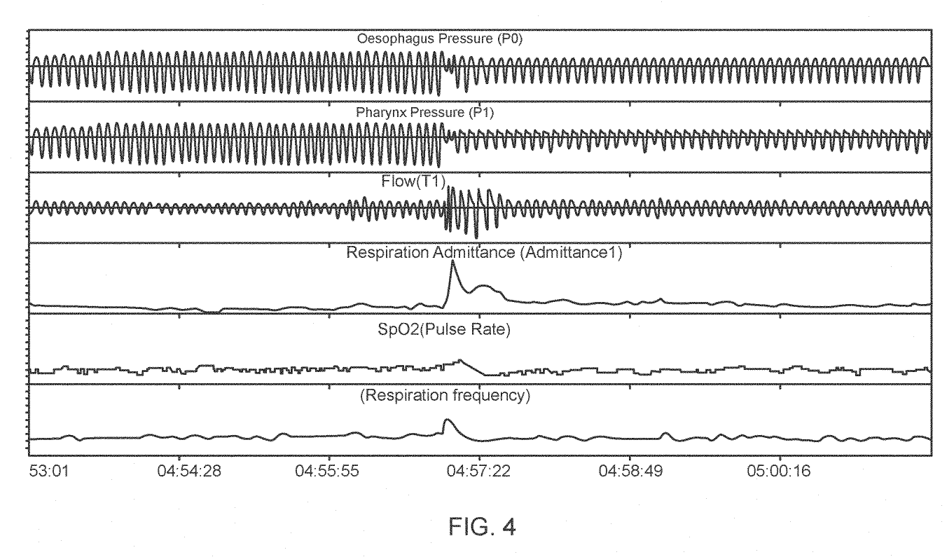

FIG. 4 shows one RERA event in detail. Prior to the RERA event pressure signals showed constant increases in amplitude while the flow (T1) amplitude was low. At 04.57 the constant increase in the pressure signals were suddenly interrupted by a distinct decrease in both pressure recordings concurrent with a rapid increase in respiratory flow. This was detected as a sharp increase in respiratory admittance. The admittance peak coincided with both an increase in pulse rate as well as a change in the respiratory pattern—here an increased respiratory rate. A RERA event is detected using respiratory admittance and is verified by increases in both parameters, pulse rate as well as respiration frequency.

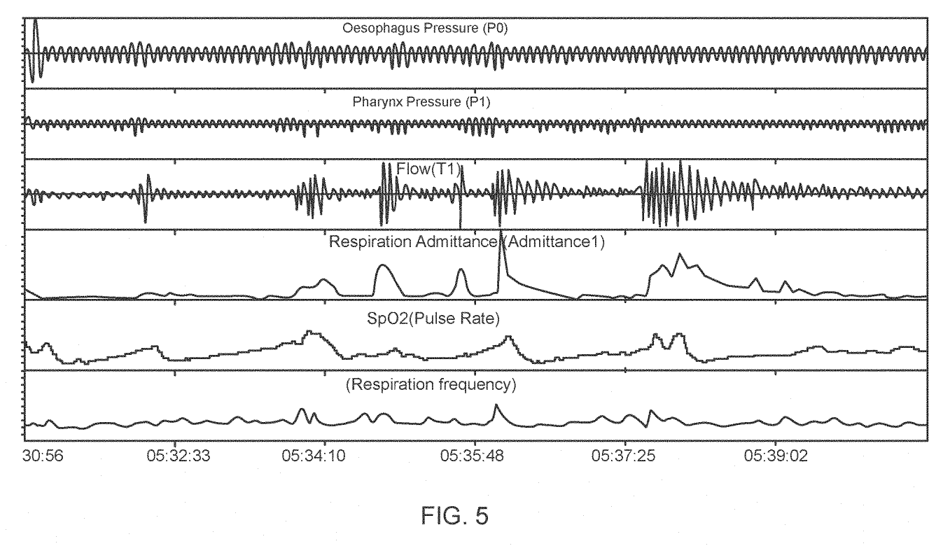

FIG. 5 demonstrates the robustness of the described algorithm. The flow signal showed a single high peak at 05:35 without any corresponding changes in the pressure recordings. In this case the admittance peaks, but without a change in pulse rate and/or respiratory rate the arousal can not be verified and this event is not rated as RERA.

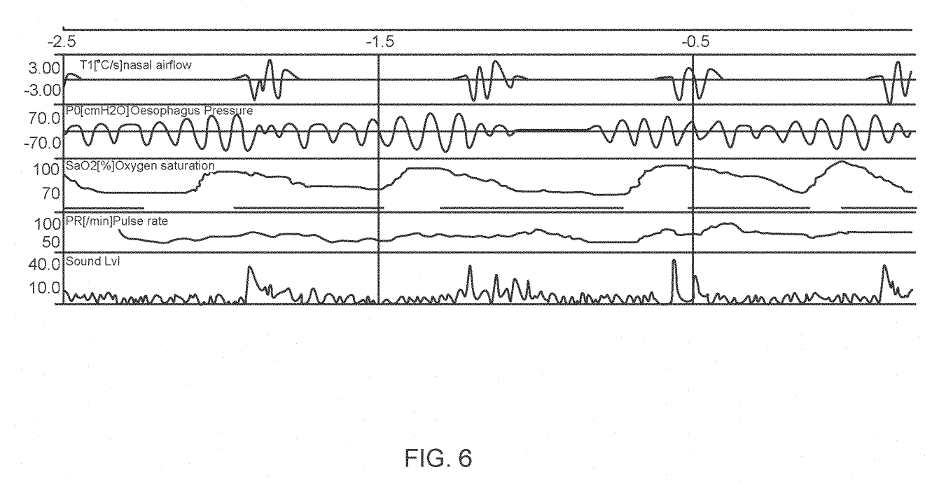

FIG. 6 shows four complete obstructions (apneas) with snoring between apneas (inter-apneic snoring): During apneas (flat line in respiratory flow T1) no snoring sounds occurred (flat line in sound Lv1) as the airways were completely blocked. After an arousal the airways opened up and breathing resumed (high amplitudes in respiratory flow T1). Concurrent with the resumed airflow the snoring signal showed high peaks.

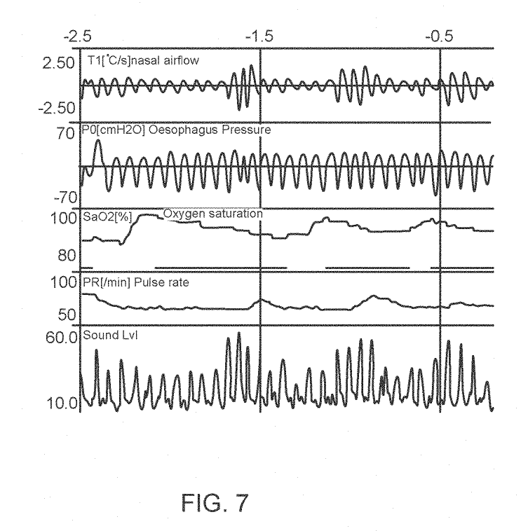

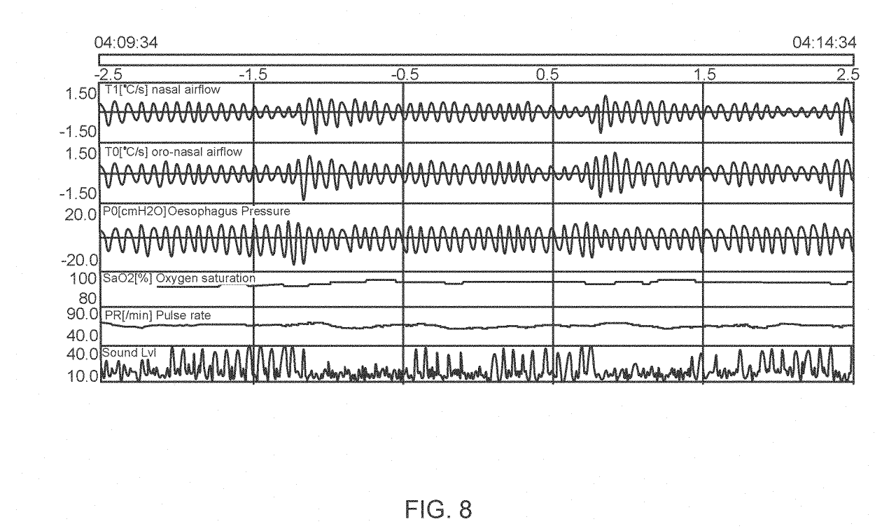

FIG. 7 shows three events with snoring during incomplete obstructions (here: hypopnea): During the hypopneas the respiratory flow showed a reduction in amplitude (T1) while the snoring persisted during the event (peaks in sound Lv1). After the arousal, both the amplitude in respiratory flow (T1) as well as the intensity of snoring (high peaks in sound Lv1) increased. During incomplete obstructions continuous snoring sounds occurred which either persisted during the arousal (FIG. 7) or diminished (FIG. 8).

FIG. 8 shows three events with snoring during incomplete obstruction (here: snoring with arousal). During the obstructive event the respiratory flow showed mild reduction in amplitude (T1, T0) with progressively higher oesophageal pressure (p0), while the snoring persisted during the event (peaks in sound Lv1). After the arousal, the amplitude in respiratory flow (T1, T0) increased, but snoring sounds were absent (flat line in sound Lv1).

Thus to summarize the invention relates to a system for monitoring respiratory effort in a patient, comprising at least one pressure sensor, the pressure sensor being positioned in a catheter and being adapted for positioning in a chosen position in the oesophagus, the pressure sensor being adapted to monitor the pressure difference between the oesophagus and external pressure at a chosen rate.

The system comprising flow measuring means for measuring the respiratory flow and analyzing means for calculating the respiratory admittance or the respiratory impedance as well as detecting the respiratory related arousals.

The system may also include means for measuring the heart rate or pulse rate, said analyzing means being adapted to correlate the calculated admittance or impedance with variations in said pulse or heart rate.

In addition the system may include means for measuring sound, especially snoring sounds, said analyzing means being adapted to correlate the calculated admittance or impedance as well as detecting the respiratory related arousals with variations in said sound.

The system may also include means for measuring respiration frequency or rate, said analyzing means being adapted to correlate the calculated admittance or impedance with variations in said respiration frequency or rate.

The analyzing means may be adapted to analyze the change in respiratory admittance per time unit that signals a respiratory event, hence the scoring parameter is the time derivative of the respiratory admittance, and/or be adapted to analyze the change in respiratory impedance per time unit that signals a respiratory event, hence the scoring parameter is the time derivative of the respiratory impedance. The analysis may for example be using accumulated experimental data, simulations or predefined models and comparing these with the measurements performed on the patient.

The sensors may be connected to a communication unit 1 on the patient for communicating the signals from said sensors to said analyzing means, which may be positioned in a computer. The computer may be a local station, even integrated in the equipment placed on the patient or connected through a network. The communication unit may use a wireless communication means or be connected through optical or electrical conductors, and may utilize internal storage and download to said analyzing means for post-recording analysis.

### LIST OF REFERENCES

1. American Academy of Sleep Medicine. ASSM Manual for the Scoring of Sleep and Associated Events. Rules, Terminology and Technical Specifications. Westchester, Ill., 2007.
2. Guilleminault C. Obstructive sleep apnea syndrome. A review. Psych Clin North Am. December 1987; 607-12.
3. Nieto F J, Young T B, Lind B K, et al. Association of sleep-disordered breathing, sleep apnea and hypertension in a large community-based study. Sleep Heart Health Study. JAMA 2000; 283:1880-81.
4. Guilleminault, C., Stoohs, R., Clerk, A., Cetel M. and Maistros, P. A cause of excessive daytime sleepiness. The upper airway resistance syndrome. Chest 1993; 104:781-7.
5. He, J. et al. Mortality and apnea index in obstructive sleep apnea. 6. Hoffman, Andrew. System for measuring respiratory function. US Pat. No. 6,287,264 B1.
7. Bradley, Donald Carmon. Method and device for determining the location of snoring. US Patent Application US2008/0249428 A1.
8. Gjersoe, B. Internal registration of gas/air-and other fluid flows in a human body and use of pressure sensors for such registration. U.S. Pat. No. 6,216,702 B1
9. Iber, C., Ancoli-Israel, S. and Chesson, A. and Quan, S. F. for the American Academy of Sleep Medicine. The AASM Manual for the Scoring of Sleep and Associated Events: Rules, Terminology and Technical Specifications, American Academy of Sleep Medicine, Westchester, Ill., 2007.
10. Katz E S, Lutz J, Black C and Marcus, C, Pulse Transit Time as a Measure of Arousal and Respiratory Effort in Children with Sleep-Disordered Breathing. Pediatric Research (2003) 53, 580-588.
11. Lavie P, Shlitner A, Sheffy J, Schnall R P. Peripheral Arterial Tonometry: A novel and sensitive non-invasive monitor of brief arousals during sleep. IMAJ 2000; 2(3):246-7.
12. Munir M. Sharif and Ahmed S. BaHammam Sleep estimation using BodyMedia's SenseWear™ armband in patients with obstructive sleep apnea Ann Thorac Med. 2013 January-March; 8(1): 53-57.
13. Sforza E; Pichot V; Cervena K et al. Cardiac variability and heart rate increment as a marker of sleep fragmentation in patients with a sleep disorder: a preliminary study. Sleep 2007; 30(1):43-51.
14. Stoohs R, Guilleminault C. Cardiovascular changes associated with the obstructive sleep apnea syndrome. J Appl Physiol. 1992; 75:583-589.
15. Geerts B F; Leon P A; Jansen J R Methods in pharmacology: measurement of cardiac output. B J Clin Pharmaco12011; 71:3 316-330.
16. Woltljer H H, Bogaard H J, de Vries P M J M The technique of impedance cardiography. Eur Heart J 1997; 18: 1396-1403.
17. To K W, Chan W C, Chan T O, Tung A, Ngai J, Ng S, Choo K L, Hui D S. Validation study of a portable monitoring device for identifying OSA in a symptomatic patient population. Respirology 2009 14(2):270-275.
18. U.S. patent application Ser. No. 10/920,675 Lee et al
19. Davies R J O, Vardi-Visy K, Clarke M, Stradling J R. Identification of sleep disruption and sleep disordered breathing from the systolic blood pressure profile. Thorax 1993; 48: 1242-7.
20. Delessert A; Espa F; Rossetti A; Lavigne G; Tafti M; Heinzer R. Pulse wave amplitude drops during sleep are reliable surrogate markers of changes in cortical activity. SLEEP 2010; 33(12):1687-1692.
21. Wheatley J; White D The influence of sleep on pharyngeal reflexes. Sleep 1993; 16(8 Supl): 87-9.
22. Matthew O P Upper airway negative pressure effects on respiratory activity of upper airway muscles. J Appl Physiol 1984; 56: 500.
23. White D Pathogenesis of Obstructive and Central Sleep Apnea. Am J Respir Crit Care Med 2005; 172: 1363-1370.
24. Morgenstern et. al. An invasive and a noninvasive approach for the automatic differentiation of obstructive and central hypopneas. IEE Trans. Biomed. Engineering. Vol.57. No 8, August 2010.
25. Bloch K E, Li Y, Sackner M A, Russi E W. Breathing pattern during sleep disruptive snoring. Eur Respir J 1997; 10: 576-586.
26 Ben-Israel N; Tarasiuk A; Zigel Y. Obstructive apnea hypopnea index estimation by analysis of nocturnal snoring signals in adults. SLEEP 2012; 35(9):1299-1305.

1. A system for monitoring respiratory effort in a patient and detecting Respiratory Effort Related Arousals (RERA) comprising means for measuring lung pressure at a certain rate,the system comprising:
flow measuring means for measuring the respiratory flow; and
analyzing means for monitoring the measured respiratory admittance and lung pressure, wherein an increase in said admittance as well as said measured lung pressure and thus detecting respiratory effort related arousals.

2. The system according to claim 1, comprising means for measuring the heart rate, said analyzing means being adapted to correlate the calculated admittance or impedance with variations in said pulse or heart rate.

3. The system according to claim 1, comprising means for measuring sound, said analyzing means being adapted to correlate the calculated admittance or impedance with variations in said sound.

4. The system according to claim 1, comprising means for measuring respiration frequency, said analyzing means being adapted to correlate the calculated admittance or impedance with variations in said respiration frequency.

5. The system according to claim 1, wherein said analyzing means is adapted to analyze the change in respiratory admittance per time unit that signals a respiratory event, wherein the scoring parameter is the time derivative of the respiratory admittance.

6. The system according to claim 1, wherein said analyzing means is adapted to analyze the change in respiratory impedance per time unit that signals a respiratory event, wherein the scoring parameter is the time derivative of the respiratory impedance.

7. The system according to claim 1, wherein said sensors are connected to a communication unit on the patient for communicating the signals from said sensors to said analyzing means being positioned in a computer.

8. The system according to claim 7, wherein said communication unit utilizes a wireless communication means.

9. The system according to claim 7, wherein said communication unit utilizes internal storage and download to said analyzing means for post-recording analysis.

10. The system according to claim 1, wherein the means for measuring lung pressure includes a catheter with a pressure sensor in the oesophagus.

11. The system according to claim 1, wherein the means for measuring lung pressure comprises one or more respiratory belts.

12. A method for detecting Respiratory Effort Related Arousals (RERA) in a patient including means for means for measuring lung pressure and flow measuring means for measuring the respiratory flow, wherein the measured respiratory admittance and lung pressure is analyzed, and generating a signal indicating a RERA when detecting an increase in admittance preceded by an increase of lung pressure.
